(19) United States
(12) Patent Application Publication
(10) Pub. No.: US 2011/0153197 A1
(43) Pub. Date: Jun. 23, 2011

(54) INSOLE TYPE NAVIGATION APPARATUS AND OPERATION METHOD THEREOF

(75) Inventor: Sa Kwang Song, Daejeon (KR)

(73) Assignee: Electronics and Telecommunications Research Institute, Daejeon (KR)

(21) Appl. No.: 12/860,744

(22) Filed: Aug. 20, 2010

(30) Foreign Application Priority Data
Dec. 21, 2009 (KR) 10-2009-0128369

(51) Int. Cl. G01C 21/34; G01S 19/42; A61B 5/103

(52) U.S. Cl. 701/201; 702/139

(57) ABSTRACT

An insole type navigation apparatus for indicating the direction of a user by interworking with a navigation device providing a road guidance service, includes: a GPS module mounted on the user's shoes and providing the user's location information to the navigation device; a plurality of vibration motors mounted in the insole of the shoes and indicating the recommended direction of the user's travel through vibrations; and a controller selectively driving vibration motors corresponding to each direction to inform the user about the direction of a destination, when the navigation device provides the road guidance service.

### CROSS-REFERENCE TO RELATED APPLICATIONS

This application claims the priority of Korean Patent Application No. 10-2009-0128369 filed on Dec. 21, 2009, in the Korean Intellectual Property Office, the disclosure of which is incorporated herein by reference.

### BACKGROUND OF THE INVENTION

1. Field of the Invention

The present invention relates to an insole type navigation apparatus and operation method thereof and, more particularly, to an insole type navigation apparatus capable of indicating a movement direction at a current location for elderly people, the visually impaired, or those who lack a sense of location, and the like, by using an insole vibration motor, rather than providing a map and an indication message on a screen as in the case of a general navigation device.

2. Description of the Related Art

As the use of the global positioning system (GPS) as a location technique is advancing, a navigation service providing information regarding the location of an object or a person, or information regarding a desired travel route has received a great deal of attention. Map matching methods developed so far are mostly for GPS navigation systems, and a map matching method fitting the characteristics of a pedestrian navigation system is yet to be developed.

Meanwhile, a pedestrian navigation system refers to a system capable of accurately providing information regarding a user's location on a digital map by using various location measurement techniques to thereby provide various location-based services (LBSs), as well as a route search, to pedestrians.

The rapid increase in the number of elderly people in many nations has prompted the development of numerous services and systems for elderly people, whose perception ability and machine manipulation capabilities, in general, steadily decline with age. In particular, there are a growing number of elderly or visually impaired people who may find it difficult to see a map or utilize a navigation device; however, existing navigation devices are mostly vehicle-oriented products which are generally inconvenient for use by pedestrians. In addition, it is not easy to apply a method in which the user is audibly instructed while viewing the screen of a navigation device to the elderly and visually impaired.

Also, a navigation scheme in a surefooted step mode (namely, a navigation device commonly used while a user is walking) is used for some youths in association with a PDA, a phone, and the like. In this case, however, in consideration of user convenience, a method for allowing the elderly and disabled to be guided along a road without viewing a screen will be very useful.

### SUMMARY OF THE INVENTION

An aspect of the present invention provides an insole type navigation apparatus for providing a navigation function to footwear to allow people who are vulnerable to new technology, such as the elderly, the visually impaired, the hearing impaired, and so on, to easily use it, thus maximizing user convenience and supplementing the shortcomings of the existing navigation device, and an operation method thereof.

Another aspect of the present invention provides an insole type navigation apparatus including a vibration device mounted inside footwear (shoes) in order to provide road guidance to users, without a voice or screen image configuration, such that only the users recognize guidance while others cannot, thus providing personal psychological stability and satisfaction, and an operation method thereof.

Another aspect of the present invention provides an insole type navigation apparatus for analyzing foot pressure distribution and adjusting road guidance according to a user's motion such as whether a user is moving, sitting, standing, and so on, thus providing a useful road guidance function, and an operation method thereof.

Another aspect of the present invention provides an insole type navigation apparatus for automatically determining a point of time at which road guidance is to be provided, upon detecting the condition of a user and providing road guidance based on the determination of time, thus increasing user convenience, and an operation method thereof.

According to an aspect of the present invention, there is provided an insole type navigation apparatus for indicating the direction of a user by interworking with a navigation device providing a road guidance service, including: a GPS module mounted on the user's shoes and providing a user's location information to the navigation device; a plurality of vibration motors mounted in the insole of the shoes and indicating the recommended direction of the user's travel through vibrations; and a controller selectively driving vibration motors corresponding to each direction in order to inform the user about the direction of a destination, when the navigation device provides the road guidance service.

According to an aspect of the present invention, there is provided an operation method of an insole type navigation apparatus for guiding to a user along a road by interworking with a navigation device providing a road guidance service, including: providing the road guidance service based on the user's location information; and when the navigation device provides the road guidance service, selectively driving a plurality of vibration motors mounted in an insole of the user's shoes to inform the user about the recommended direction of the user's travel to a destination.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 illustrates the configuration of an insole type road guidance system according to an exemplary embodiment of the present invention;

FIG. 2 illustrates a detailed configuration of the insole type road guidance system according to an exemplary embodiment of the present invention;

FIG. 3 is a schematic block diagram showing the configuration of an insole type navigation apparatus according to an exemplary embodiment of the present invention;

FIG. 4 is a flow chart illustrating the process of an operation method of the insole type navigation apparatus according to an exemplary embodiment of the present invention; and

FIG. 5 is a flow chart illustrating the process of a method for detecting a user's current condition by using a user's foot pressure data according to an exemplary embodiment of the present invention.

### DETAILED DESCRIPTION OF THE PREFERRED EMBODIMENT

Exemplary embodiments of the present invention will now be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art. In the drawings, the shapes and dimensions may be exaggerated for clarity, and the same reference numerals will be used throughout to designate the same or like components.

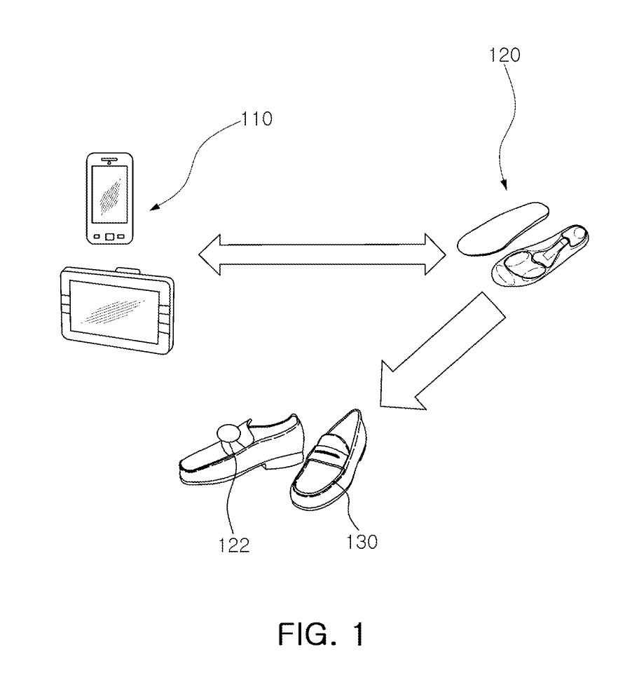

FIG. 1 illustrates the configuration of an insole type road guidance system according to an exemplary embodiment of the present invention.

With reference to FIG. 1, the insole type road guidance system according to an exemplary embodiment of the present invention includes a navigation device 110 and an insole type navigation apparatus 120 interworking with the navigation device 110 through a wireline or wirelessly.

The navigation device 110 may be a mobile phone, a PDA, a navigation system, and the like, and provides a road guidance service to users through a screen image and a sound.

The insole type navigation apparatus 120 is mounted in general shoes 130, receives a user's location information through the GPS module 122 mounted on an upper portion of the shoes 130, and when the navigation device 110 provides a road guidance service, the insole type navigation apparatus 120 selectively drives vibration motors corresponding to each direction to inform the user about the direction of a destination.

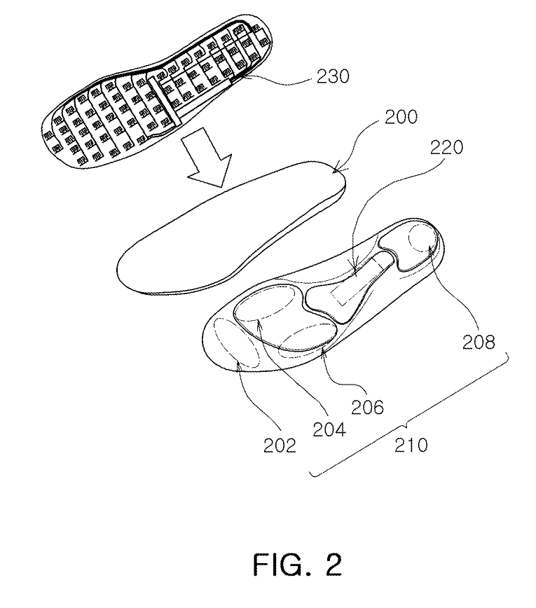

FIG. 2 illustrates a detailed configuration of the insole type road guidance system according to an exemplary embodiment of the present invention.

With reference to FIG. 2, the insole type navigation device 210 includes a plurality of vibration motors 210, a controller 220, and the like.

The plurality of vibration motors 210 are mounted on four areas 202, 204, 206, and 208 of the bottom surface of the insole 200. The respective vibration motors vibrate to guide a user in four directions, including up, down, right, and left, and two vibration motors operate simultaneously to guide the directions such as in a left upward direction, a left downward direction, a right upward direction, and a right downward direction.

The controller 220 is provided on the bottom surface of the insole 220 along with the plurality of vibration motors 210 and interworks with the navigation device 110 via a wireline/wireless communication unit (not shown), and when the navigation device 110 provides a road guidance service, the controller 220 selectively drives the vibration motors 210 corresponding to each direction to inform the user about the direction of a destination.

Also, the insole type navigation device 120 further includes a plurality of pressure sensors 230.

The plurality of pressure sensors 230 are mounted on a front surface of the insole 200 in order to detect a user's foot pressure data. In an exemplary embodiment of the present invention, fifty eight pressure sensors are used to detect the user's foot pressure data.

The controller 220 detects the user's current condition including standing, sitting, raising his foot, or the like, by using the users foot pressure data, and controls the operation of the plurality of vibration motors 210 according to the user's current condition.

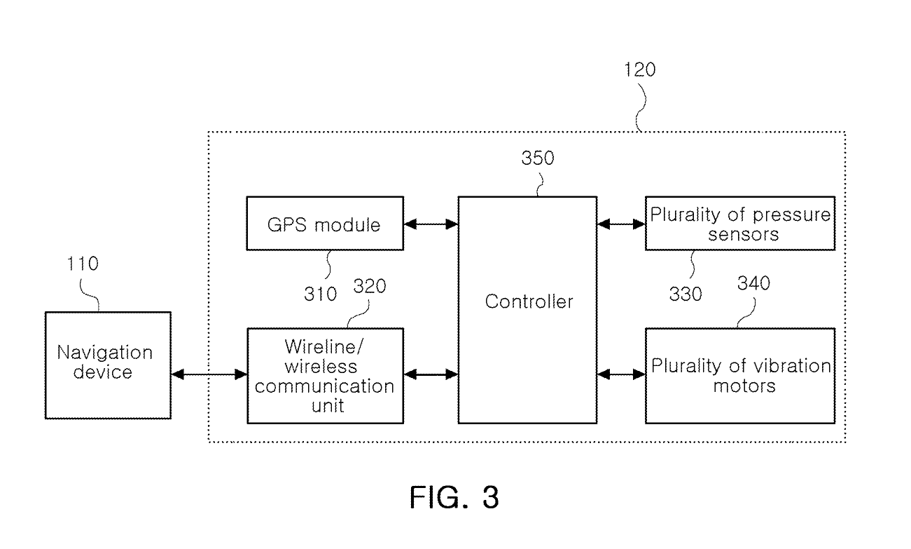

A detailed description of the operation of the insole type navigation apparatus 120 will now be described with reference to FIG. 3.

FIG. 3 is a schematic block diagram showing the configuration of an insole type navigation apparatus according to an exemplary embodiment of the present invention.

With reference to FIG. 3, the insole type navigation apparatus 120 according to an exemplary embodiment of the present invention includes a GPS module 310, a wireline/wireless communication unit 320, a plurality of pressure sensors 330, a plurality of vibration sensors 340, a controller 350, and the like.

The GPS module 310 is mounted on the user's shoes, receives the user's location information, and provides the received location information to the external navigation device 110.

The wireline/wireless communication unit 320 allows the insole type navigation apparatus 120 to interwork with the external navigation device 110 through short-range wireless communications methods such as WLAN, Bluetooth™, ZigBee™, and the like.

The plurality of pressure sensors 330 are mounted on the bottom surface of the insole 200 in order to detect a user's foot pressure data.

The plurality of vibration motors 340 are mounted on the four areas of the bottom surface of the insole 200. The vibration motors vibrate to guide four directions including up, down, left, and right, and vibration motors at two positions simultaneously operate to guide the directions such as a left upward direction, a left downward direction, a right upward direction, and a right downward direction.

When the external navigation device 110 provides a road guidance service, the controller 350 selectively drives the vibration motors 340 corresponding to each direction to inform the user about the direction in which to travel to reach a destination. For example, when the destination is to the right, the vibration motor mounted at the right side of the bottom surface of the insole 200 vibrates to inform the user that he is supposed to go to the right.

Also, the controller 350 detects the user's current condition by using the user's foot pressure, and controls the operation of the plurality of vibration motors 340 according to the user's current condition. Namely, when the user moves, the controller 350 controls the plurality of pressure sensors 330, commanding them to operate, and when the user does not move, the controller 350 controls the plurality of pressure sensors 330, commanding them not to operate.

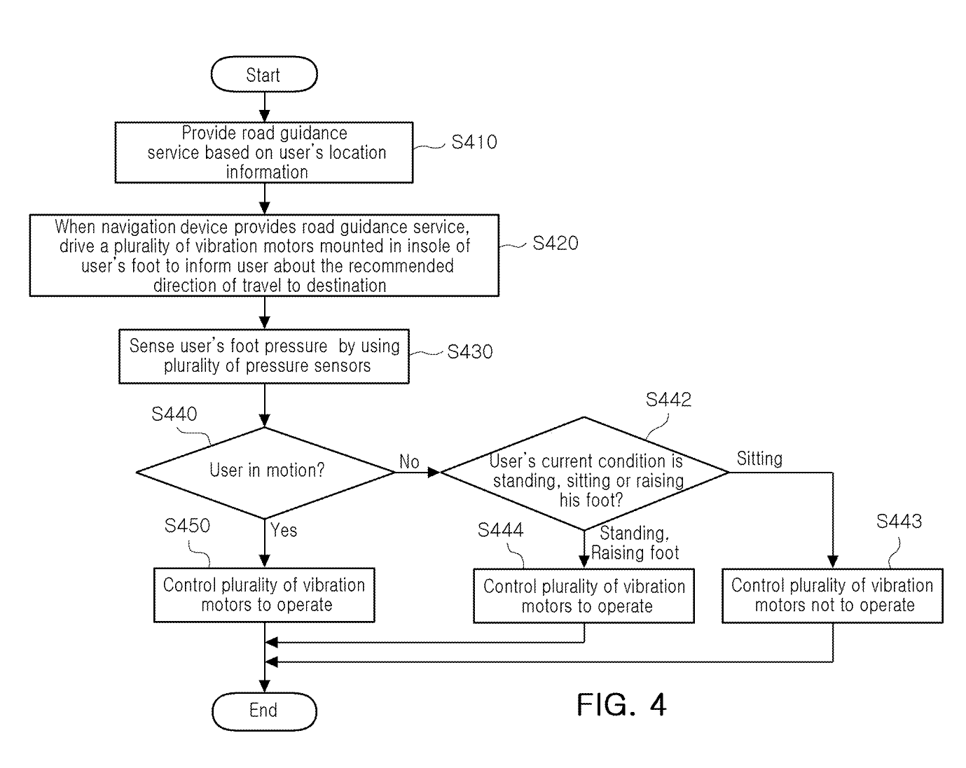

FIG. 4 is a flow chart illustrating the process of an operation method of the insole type navigation apparatus according to an exemplary embodiment of the present invention.

With reference to FIG. 4, a road guidance service is provided based on information regarding the user's location (S410).

When the navigation device 110 provides the road guidance service, the plurality of vibration motors 340 mounted in the insole 220 of the user's shoes are selectively driven to inform the user about the direction of his destination (S420). In this case, the directions of the destination are informed by using eight directions including an upward direction, a downward direction, a leftward direction, a rightward direction, a left upward direction, a left downward direction, a right upward direction, and a right downward direction.

Subsequently, a user's foot pressure data is sensed by using the plurality of pressure sensors 330 mounted in the insole 200 (S430).

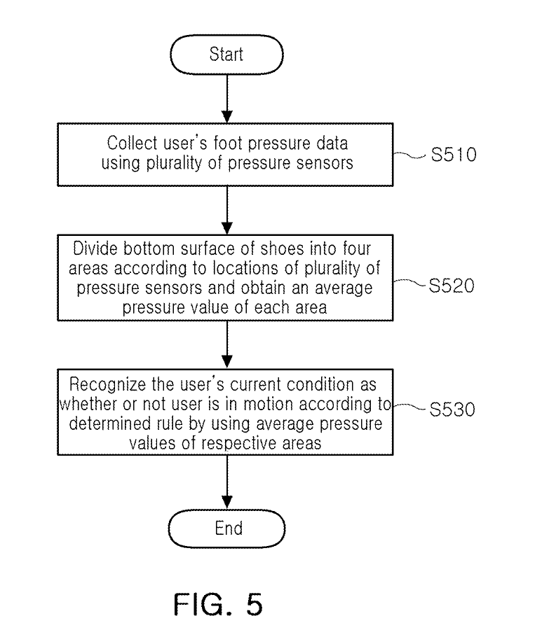

And then, the user's current condition is detected by using the user's foot pressure data, and it is determined whether or not the user is moving (S440). The detailed method of detecting the user's current state will be described later with reference to FIG. 5.

When the user is moving, the plurality of vibration motors 340 are controlled to operate (S450).

When the user is not moving, it is determined whether or not the user's current condition is standing, sitting, or raising a foot (S4420). For example, within the range of pressure values 0 to 1000 (the current pressure sensor has a smaller value as the pressure increases), 300 as a reference pressure value for standing and sitting, while 600 as a reference pressure value for sitting and raising a foot. If the overall average pressure value is 240, it is recognized as standing, if it is 450, it is recognized as sitting, and if it is 600, it is recognized as raising a foot.

When the user's current condition is standing or raising his foot, the plurality of vibration motors 340 are controlled so as to operate (S444), and when the user's current condition is sitting, the plurality of vibration motors 340 are controlled so as not to operate (S443).

FIG. 5 is a flow chart illustrating the process of a method for detecting a user's current condition by using a user's foot pressure data according to an exemplary embodiment of the present invention.

With reference to FIG. 5, first, foot pressure data is gathered by using the plurality of pressure sensors 330 (S510).

The bottom surface of the user's shoes are divided into four areas according to the positions of the plurality of pressure sensors 330 and an average pressure value of each area is obtained (S520). Namely, the bottom surface of the user's shoes are divided into four areas including a left upper portion, a right upper portion, a left lower portion, and a right lower portion according to the positions of fifty eight pressure sensors.

Subsequently, whether or not the user is moving is recognized according to a predetermined rule by using the average pressure values of each area (S530). For example, when the order of the sizes of representative values (i.e., average pressure values) of the four areas is reversed by certain time unit (e.g., two seconds), it is recognized that the user is moving, while if the order of the sizes of the representative values of the four areas is not reversed, it is recognized that the user is not moving.

In this manner, in the exemplary embodiment of the present invention, road guidance can be provided while the user undertakes certain behaviors (e.g., motion, standing, and the like), whereby road guidance can be provided in consideration of the user's situation.

As set forth above, according to exemplary embodiments of the invention, because road guidance is provided to the elderly, the hearing impaired or the visually impaired through shoes which are constantly worn, the convenience of portability and affinity can be provided. Also, because only the user can sense vibrations, the user's confidence, psychological satisfaction and satisfaction can be improved.

In addition, because a novel road guidance scheme is provided, not aiming at substituting the existing navigation system, it can easily interwork with existing products so as to be commercialized.

Moreover, because actual modules for road guidance are mounted in the insole, only the insole may be altered according to a personal preference, increasing a ripple effect in a daily life.

Furthermore, the insole type navigation apparatus can be utilized for the road guidance of senior citizens who live alone, the elderly with dementia, children, and so on, reducing the burden on caregivers and the cost of providing social welfare services in the wake of aging.

While the present invention has been shown and described in connection with the exemplary embodiments, it will be apparent to those skilled in the art that modifications and variations can be made without departing from the spirit and scope of the invention as defined by the appended claims.

What is claimed is:

1. An insole type navigation apparatus for indicating the direction of a user by interworking with a navigation device providing a road guidance service, the apparatus comprising:
a GPS module mounted on the user's shoes and providing the user's location information to the navigation device;
a plurality of vibration motors mounted in the insole of the shoes and indicating the recommended direction of the user's travel through vibrations; and
a controller selectively driving vibration motors corresponding to each direction to inform the user about the direction of a destination, when the navigation device provides the road guidance service.

2. The apparatus of claim 1, further comprising:
a plurality of pressure sensors mounted in the insole of the shoes to sense the user's foot pressure data,
wherein the controller detects the user's current condition by using the user's foot pressure data and controls the operation of the plurality of vibration motors according to the user's current condition.

3. The apparatus of claim 2, wherein the controller collects user's foot pressure data by using the plurality of pressure sensors, divides the bottom of the shoes into four areas according to the locations of the plurality of pressure sensors to obtain an average pressure value of each area, and recognizes the user's current condition as whether or not the user is in motion according to a change of sizes of the average pressure values of each area.

4. The apparatus of claim 3, wherein when the user is in motion, the controller controls the plurality of vibration motors to operate.

5. The apparatus of claim 3, wherein when the user is not moving, the controller discriminates the user's current condition into standing, sitting, and raising his foot.

6. The apparatus of claim 5, wherein when the user's current condition standing or raising his foot, the controller controls the plurality of vibration motors so as to operate.

7. The apparatus of claim 5, wherein when the user's current condition is sitting, the controller controls the plurality of vibration motors so as not to operate.

8. The apparatus of claim 1, wherein the plurality of vibration motors are mounted in four directions including up, down, left, and right of the insole of the shoes, and indicates eight directions including up, down, left, right, left up, left down, right up, and right down.

9. An operation method of an insole type navigation apparatus for guiding a user along a road by interworking with a navigation device providing a road guidance service, the method comprising:
providing the road guidance service based on the user's location information; and
when the navigation device provides the road guidance service, selectively driving a plurality of vibration motors mounted in an insole of the user's shoes to inform the user about the recommended direction of travel to a destination.

10. The method of claim 9, wherein, informing the user about the recommended direction is indicating eight directions including up, down, left, right, left up, left down, right up, and right down with respect to the bottom surface of the shoes.

11. The method of claim 9, further comprising:
sensing a user's foot pressure data by using a plurality of pressure sensors mounted in an insole of the shoes;
detecting the user's current condition by using the user's foot pressure data; and
controlling the operation of the plurality of vibration motors according to the user's current condition.

12. The method of claim 11, wherein the detecting of the user's current condition comprises:
collecting user's foot pressure data by using the plurality of pressure sensors;
dividing the bottom surface of the shoes into four areas according to the locations of the plurality of pressure sensors and obtaining an average pressure value of each area; and
recognizing the user's current condition as whether or not the user is in motion according to a change of the sizes of the average pressure values of the respective areas.

13. The method of claim 11, wherein the controlling of the operation of the plurality of vibration motors comprises: when the user is in motion, controlling the plurality of vibration motors to operate.

14. The method of claim 11, wherein the controlling of the operation of the plurality of vibration motors comprises:
when the user is not moving, determining whether or not the user's current condition is standing, sitting, or raising his foot; and
when the user's current condition is standing or raising his foot, controlling the plurality of vibration motors so as to operate.

15. The method of claim 14, further comprising:
when the user's current condition sitting, controlling the plurality of vibration motors so as not to operate.
